(19) United States
(12) Patent Application Publication
(10) Pub. No.: US 2020/0069481 A1
(43) Pub. Date: Mar. 5, 2020

(54) Method Of Making An Absorbent Article With Reactive Compositions

(71) Applicant: The Procter & Gamble Company, Cincinnati, OH (US)

(72) Inventors: Stefan Roell, Bensheim (DE); Anupam Sinha, Schwalbach am Taunus (DE)

(21) Appl. No.: 16/118,483

(22) Filed: Aug. 31, 2018

(51) Int. Cl. A61F 13/15; A61F 13/42; A61B 5/00

(52) U.S. Cl. CPC A61F 13/15577; A61F 13/42; G01D 9/005; A61F 2013/424; A61B 5/6808

(57) ABSTRACT

A method of creating a disposable absorbent article sample for lab testing is described. The method includes providing a disposable absorbent article, applying the template to the disposable absorbent article such that the disposable absorbent article is exposed through the open area, and wherein the open area comprises a first open section and a second open section, the first open section and the second open section being discontinuous from one another, spraying reactive composition on the template covering the open area or areas.

### FIELD OF THE INVENTION

The present invention pertains to methods of creating disposable absorbent articles with reactive materials.

### BACKGROUND OF THE INVENTION

Disposable absorbent articles are utilized by consumers to manage fluids that are to be contained in a confined space. Some examples of disposable absorbent articles include sanitary towels, baby diapers, baby pants, liners, sanitary pads, sweat bands and protective clothes, pads or pants for urinary incontinence.

Performance assessment of disposable absorbent articles is often done to verify, validate or compare products. Some useful performance metrics are with regard to speed of absorption, wetness on the surface of the product, rewet, or liquid run-off, etc. Another metric which can be important is with regard to comfort of the article during use. Some metrics which are useful for describing the comfort of disposable absorbent articles are temperature during use, relative humidity during use, and pH during use, etc. Unfortunately, configuring sample disposable absorbent articles for performance assessments can be arduous. For example, conductive materials, e.g. wires, may need to be attached to a topsheet of an experimental article. For in use testing, materials would need to be selected such that the experimental absorbent article still conforms to the user while maintaining its conductivity.

Regarding the comfort metrics, often times, samples need to be prepared for human usage. And, much like the foregoing, preparation of samples for comfort metrics can also be arduous. Moreover, in many instances, some of the comfort metrics may require subjective input from the user which is difficult to reconcile amongst panelists.

Based on the foregoing, there exists a need for a method which facilitates the construction of disposable absorbent article samples for evaluating either performance and/or comfort metrics.

### SUMMARY OF THE INVENTION

The method of the present disclosure provides a facilitated pathway for the construction of disposable absorbent articles which can be utilized in evaluating performance and/or comfort metrics. In some forms, a method for creating a disposable absorbent article for lab testing may comprise the steps of: providing a disposable absorbent article; providing a template having a land area and an open area; applying the template to the disposable absorbent article such that the disposable absorbent article is exposed through the open area, and wherein the open area comprises a first open portion and a second open portion, the first portion and the second portion being discontinuous from one another; spraying a conductive composition over the template such that the conductive composition contacts the disposable absorbent article; and removing the template from the disposable absorbent article.

### BRIEF DESCRIPTION OF THE DRAWINGS

FIG. 1 is a schematic illustration showing an exemplary template for application to a disposable absorbent article.

FIG. 2 is a schematic illustration showing the template of FIG. 1 applied to a disposable absorbent article and a reactive composition being sprayed thereon.

FIG. 3A is a schematic illustration showing a disposable absorbent article post the spray process depicted in FIG. 2.

FIG. 3B is a schematic illustration showing a closeup view of section 3B highlighted in FIG. 3A.

FIG. 4 is a schematic illustration showing another embodiment of a disposable absorbent article post the reactive composition deposition described herein.

FIG. 5A is a schematic illustration showing another exemplary template for application to a disposable absorbent article.

FIG. 5B is a schematic illustration showing another embodiment of a disposable absorbent article post the reactive composition deposition described herein.

FIG. 6 is a schematic illustration showing another exemplary template for application to a disposable absorbent article.

FIG. 7 is a schematic illustration showing a disposable absorbent article in accordance with the present disclosure in signal communication with a processing unit.

### DETAILED DESCRIPTION OF THE INVENTION

The present invention pertains to a facilitated method by which disposable absorbent articles may be created for lab or consumer testing. A bevy of information may be obtained via the absorbent articles described herein. For example, the following metrics may be measured: fluid acquisition speed, fluid distribution speed, relative humidity, temperature, blood pressure, and/or stress, etc. And with the facilitated methods described herein, one or more of these metrics may be measured for one or more absorbent articles.

The use of the terms “absorbent article” or “disposable absorbent article” are meant to refer to baby diapers, baby pants, adult incontinence pants, adult incontinence diapers, adult incontinence pads, feminine hygiene pads (including pantiliners), tampons, pessaries, interlabial products, cleaning wipes associated with these products, household care wipes, or cleaning wipes such as Swiffer™ pads. While reference will primarily be made to feminine hygiene articles herein, the description of the present invention is equally applicable to the absorbent articles as defined herein.

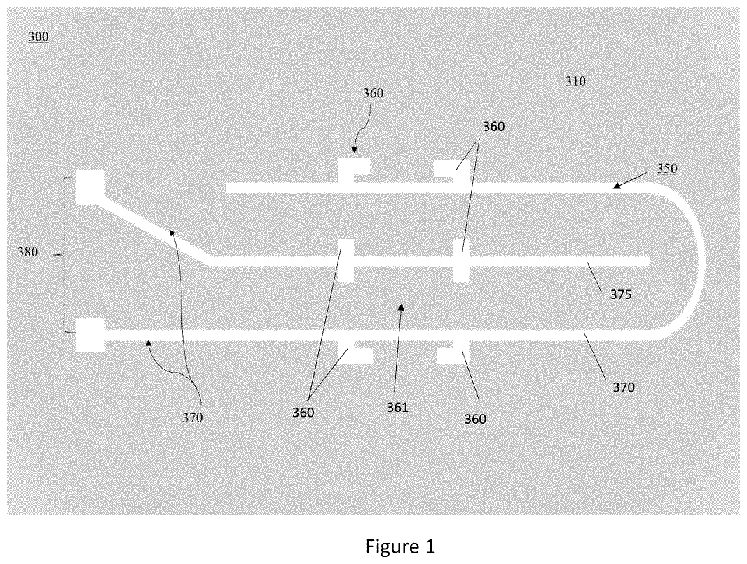

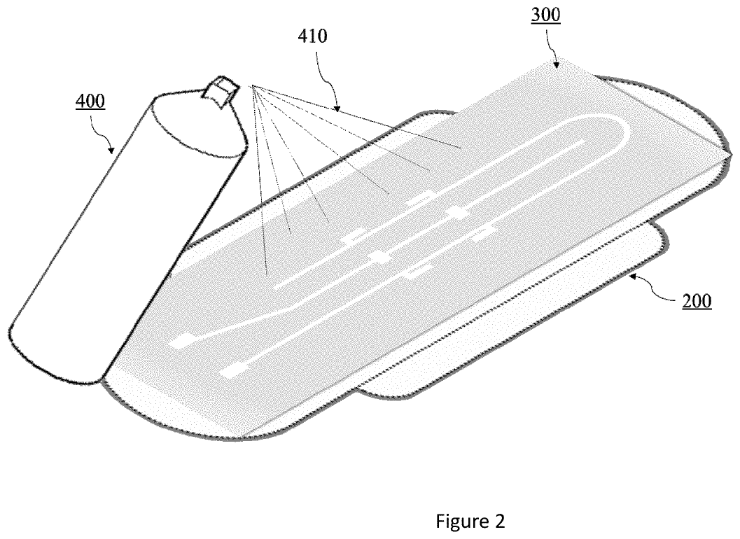

Referring to FIGS. 1 and 2, a disposable absorbent article 200 is provided for modification. As shown, the disposable absorbent article 200 is a feminine hygiene pad. However, in general, any absorbent article for which performance and/or comfort data is desired may utilize the methods of the present disclosure.

The disposable absorbent article 200 may be obtained via any suitable method. For example, where performance data and/or comfort data for competitive products is desired, the absorbent articles may be purchased from any suitable retailer. For manufacturers of disposable absorbent articles that desire performance data and/or comfort data of their own products, disposable absorbent articles may be obtained by manufacturing the products for which data is desired. Some exemplary disposable absorbent articles which can be utilized in conjunction with the methods described herein may comprise a topsheet, a backsheet, and an absorbent core disposed between the topsheet and the backsheet.

A template 300 is also provided either prior to, along with, or post the provision of the absorbent article(s). As shown, the template 300, in some forms, may comprise a land area 310 and an open area 350. The open area 350 may comprise a first open section 370 and a second open section 375 which are discontinuous from one another. The open area 350 may further comprise a pair of open contact areas 380, one of which is associated with the first open section 370, and one of which is associated with the second open section 375. As shown, portions of the first open section 370 and the second open section 375 overlap with an area of interest 361.

As shown, the open area 350 may further comprise open markers 360 which identify the area of interest 361. In some forms, as shown, the open markers 361 may form a boundary around the area of interest 361. However, the open area 350 is not required to identify the area of interest 361. In some forms, the area of interest 361 may be highlighted via ink pen, pencil, marker, tape, etc. As such, the open markers 360 may be optionally comprised by the open area 350.

The land area of the template 300 may be made of a material that does not react with the reactive composition that is applied to the template. The land area material may be, for example, a polymer which is flexible and allows cleaning after use. Some suitable exemplary materials include paper, metal films or combination of those. In some forms, the template 300 may be obtained from a manufacturer or retailer. In other forms, templates 300 may be created as desired for the desired test. For example, a desired open area may be cut into a piece of sheet metal.

Still referring to FIGS. 1 and 2, after the provision of the template 300, the template 300 is then applied to the disposable absorbent article 200 such that the disposable absorbent article 200, or portions thereof, are exposed through the open area 350 of the template 300. The template 300 should be placed on the disposable absorbent article 200 such that the open area 350 corresponds to an area of the disposable absorbent article 200 for which data is desired. For example, where the fluid acquisition data is desired near the center of the disposable absorbent article 200, the template 300 should be placed on the disposable absorbent article 200 such that the first open section 370 and the second open section 375 are associated with the center of the disposable absorbent article 200.

As shown, once the template 300 has been applied to the absorbent article 200, a reactive composition 410 may be sprayed onto the template 300 and absorbent article 200. As shown, the reactive composition 410 may be applied via a pressurized spray can 400, in some forms. The pressurized spray can 400 may be held over the template 300 while the reactive composition 410 is released. After application of reactive composition 410, the template 300 may be removed leaving reactive composition which corresponds to the open area 350 on the surface of the disposable absorbent article 200. Other forms of application of the reactive composite 410 are contemplated.

The reactive composition contained in the pressurized can may be a combination of materials with special properties in a liquid, or gaseous basis acting as a solvent. These added materials define the function of the reactive composition. Examples of the added material are carbon black, silver or aluminum particles, these particles have the property of electrical conductivity and could be used as electrical connectors as well as conductivity sensors. Any suitable material may be utilized in the reactive composition of the present disclosure. Some suitable materials include ceramic, polymers, metals or a combination of those, that reacts to, for example, mechanical, electrical, and/or chemical, stimuli and which signal generated from that reaction can be measured. Reactive compositions are further described in U.S. Pat. Nos. 8,088,315; 8,124,549; and U.S. Patent Application Publication No. 2010/0286583.

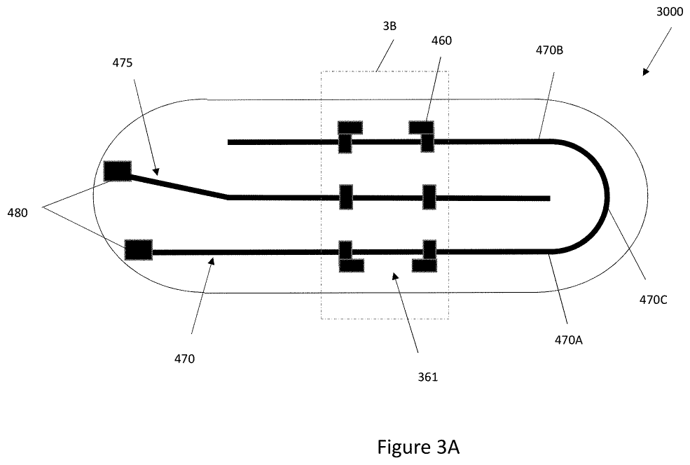

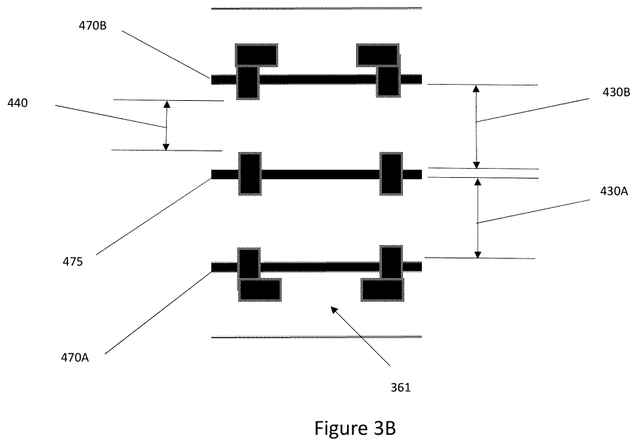

After application of the spray, the template 300 is removed from the disposable absorbent article. The sprayed reactive composition that is applied through the open area 350 remains on the disposable absorbent article as shown in FIGS. 3A and 3B. As shown, on a disposable absorbent article 3000, the reactive composition forms a first lead 470 and a second lead 475 which correspond to the first open section 370 (shown in FIG. 1) and the second open section 375 (shown in FIG. 1) in the template 300 (shown in FIG. 1), respectively. The first lead 470 comprises a first portion 470A, a second portion 470B and a joining portion 470C which joins the first portion 470A and the second portion 470B. The reactive composition also forms a pair of filled contact areas 480 which correspond to the pair of open contact areas 380 (shown in FIG. 1) of the template 300 (shown in FIG. 1). And, the reactive composition, in some forms of the present disclosure, may form a plurality of filled markers 460 which correspond to the plurality of open markers 360 (shown in FIG. 1) of the template 300 (shown in FIG. 1).

As shown, the first lead 470 comprises portions which overlap the area of interest 361, i.e. the first portion 470A and the second portion 470B. And, as shown, the second lead 475 comprises only one overlapping portion with the area of interest 361. Forms are contemplated where the second lead 475 comprises a plurality of overlapping portions in conjunction with the first lead 470 comprising a plurality of overlapping portions. Such forms are discussed in additional detail hereafter.

Depending on the metric being measured, spacing between the first portion 470A and the second lead 475, between the second portion 470B and the second lead 475, and between the second lead 475 and the joining portion 470C may be of import. For example, for those tests where liquid insults are added to the area of interest to determine distribution speed and/or acquisition speed, the spacing described above may be important. For such tests, the liquid insult over a period of time can form a conductive bridge between the first portion 470A and the second lead 475, the second lead 475 and the second portion 470B, and/or the second lead 475 and the joining portion 470C.

As shown, a first space 430A may separate inner surfaces of the first portion 470A and the second lead 475. A second space 430B may separate inner surfaces of the second portion 470B and the second lead 475. And, for those forms which comprise filled markers 460, a third space 440 may separate adjacent filled markers 460. The first space 430A and the second space 430B should be equal within the area of interest 361. Outside of the area of interest 361, the spacing between the first portion 470A and the second lead 475, between the second portion 470B and the second lead 475, and between the second lead 475 and the joining portion 470C are much less important. However, within the area of interest 361, a spacing which is too close can yield results which indicate no discernable difference amongst disposable absorbent articles because a connection is easily established between adjacent leads. In contrast, a spacing which is too high can yield no signal as the liquid insult does not have an opportunity to form a conductive bridge between the first lead 470 and the second lead 475.

Appropriate spacing between the first lead 470 and the second lead 475 can alleviate some of the variability in the data. For example, in some forms, the first space 430A may be between about 10 mm to about 20 mm, from about 12 mm to about 18 mm, from about 14 mm to about 16 mm, or the first space 430A may be about 15 mm, specifically reciting all values within these ranges and any ranges created thereby. This spacing is equally applicable for the third space 440 between adjacent filled markers 460. Generally, where higher liquid insults are utilized, a larger spacing between adjacent leads may be utilized, and where lower liquid insults are utilized, smaller spacing between adjacent leads may be appropriate.

Where the disposable absorbent articles are to be utilized in lab testing, the pattern of reactive composition depicted in FIGS. 3A and 3B may be sufficient. However, for consumer use testing, a different pattern may need to be utilized. In consumer use testing, the disposable absorbent article can bend along several different axes and may be subject to bunching. Accordingly, a more expansive grid of reactive composition may need to be utilized to ensure that adequate readings are obtained.

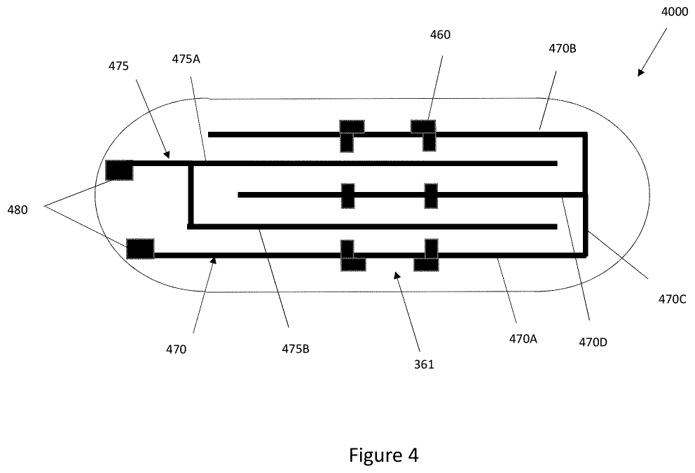

Another exemplary pattern of reactive composition on a disposable absorbent article is shown in FIG. 4. As shown a disposable absorbent article 4000 for use in consumer testing may comprise a more comprehensive reactive composition pattern than did the disposable absorbent article 3000 of FIGS. 3A and 3B. For the disposable absorbent article 4000, the first lead 470 may comprise the first portion 470A, the second portion 470B, and the joining portion 470C as described previously with regard to FIGS. 3A and 3B. However, in addition, the first lead 470 may further comprise a third portion 470D which is joined to the first portion 470A and the second portion 470B via the connecting portion 470C. The second lead 475 may comprise a first portion 475A and a second portion 475B. The spacing between the inner surface of the third portion 470D and the inner surface of the first portion 475A of the second lead 475 and the inner surface of the second portion 475B of the second lead 475 should be similar to the first space 430A and the second space 430B described previously with regard to FIG. 3B.

As shown, the reactive composition pattern shown in FIG. 4 may comprise more overlapping portions (portions of the first lead 470 and/or the second lead 475) which overlap the area of interest 361. As mentioned previously, the first lead 470 may comprise a plurality of overlapping portions. As shown, the first portion 470A, the second portion 470B and the third portion 470D each extend through and overlap the area of interest 361. And, the second lead 475, similar to the first lead 470, may comprise a plurality of overlapping portions. As shown, the first portion 475A and the second portion 475B of the second lead 475 may extend through and overlap the area of interest 361.

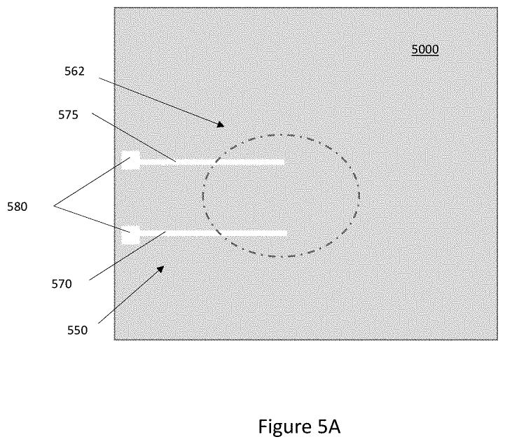

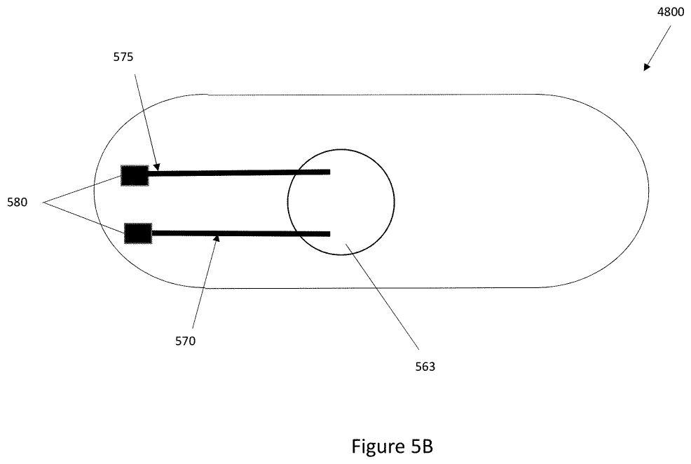

Additional configurations of reactive composition on a disposable absorbent article are contemplated. For example, referring to FIG. 5A, forms are contemplated where a template 5000 comprises an open area 550 which is associated with an area of interest 562 on a disposable absorbent article. Similar to the open area 350 (shown in FIG. 1), the open area 550 may provide open markers to highlight the area of interest 562, in some forms. And, in some forms, the area of interest 562 may correspond to a sensor which is placed in the pad. In such forms, the sensor may be placed onto or into the pad prior to the application of the template 5000. As shown, the open area 550 comprises a first open section 570 and a second open section 575 and a pair of open contact areas 580 connected to the first open section 570 and the second open section 575.

After spraying the reactive composition, as described herein, over the template 5000, a resultant disposable absorbent article 4800 is shown in FIG. 5B. As shown, the reactive composition may form a first lead 570 and a second lead 575 which are in signal communication with a pair of filled contact areas 580. The first lead 570 and the second lead 575 may connect to a sensor 563. As shown, the sensor 563 may correspond to the area of interest 562.

Any suitable sensor may be utilized. Some examples include temperature, relative humidity, position, noninvasive blood pressure sensor, and/or a pH sensor. Forms of the present invention are contemplated where a plurality of sensors may be utilized in an absorbent article. Any suitable sensor combination is contemplated. Some examples include lab samples with a relative humidity and temperature sensors and samples for consumer use testing that have relative humidity, temperature, pH and position sensors.

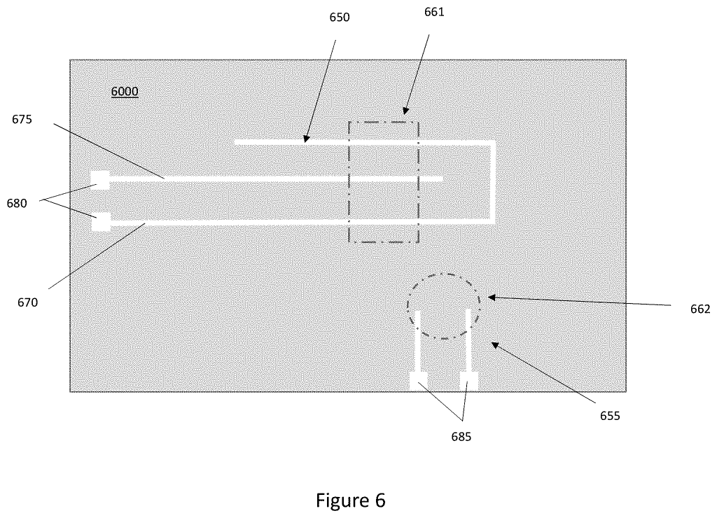

Forms of the present disclosure are also contemplated where a disposable absorbent article comprises a plurality of areas of interest which are configured as described heretofore. For example, a disposable absorbent article according to one form of the present disclosure may comprise a first pattern of reactive composition similar to that shown FIG. 3A or 4. Additionally, the disposable absorbent article may further comprise a second pattern of reactive composition which is configured similar to that shown in FIG. 5B. Such an example is discussed in additional detail with regard to FIGS. 6A and 6B.

Referring to FIG. 6, forms of the present disclosure are contemplated where a template 6000 comprises a plurality of open areas, i.e. 650 and 655. In some forms, the open area 650 may be configured as described heretofore with regard to FIG. 1 or a template which would correspond to the resultant disposable absorbent article in FIG. 4. The open area 650 may be associated with a first area of interest 661 while the second open area 655 may be associated with a second area of interest 662. In some forms, the first open area 650 may provide open markers to indicate the first area of interest 661 on the corresponding disposable absorbent article.

As shown, the first open area 650 comprises a first open section 670 and a second open section 675, which in some forms, may be configured as shown in FIG. 1. The first open section 670 and the second open section 675 connect to a pair of open contact areas 680. The second open area 655 may comprise a first open section 690 and second open section 695. The first open section 690 and the second open section 695 may connect to a pair of open contact areas 685.

Regarding the second open area 655, its configuration may be set up similar to that described heretofore regarding open area 550 (shown in FIG. 5A) in some forms. Namely, the second open area 655 may be configured such that a sensor may be utilized in a disposable absorbent article. The sensor may correspond to the second area of interest 662.

Regardless of the configuration of the reactive composition on a disposable absorbent article, after removal of the template, the sprayed disposable absorbent article may be utilized to measure desired metrics as described herein. As shown in FIG. 3A and in FIG. 4, disposable absorbent articles 3000 and 4000, respectively, comprise a pair of filled contact areas 480 and 580, respectively. These filled contact areas can serve as a connection between the areas of interest/sensors and a data recording unit.

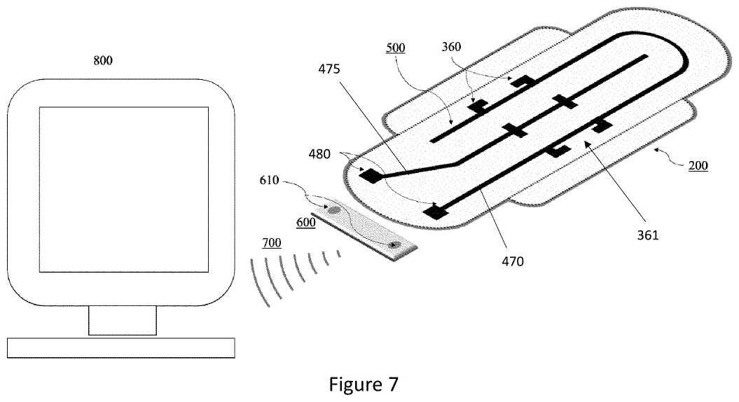

Referring now to FIG. 7, a data recording unit 600 may be in signal communication with the filled contact areas 480. The shape and size of the filled contact areas is not limited to either symmetry or shape, for example, one could be bigger than the other and have a circular area instead of rectangular. The size and shape is to be determined according to the test configuration and connections of the data recording unit 600. The filled contact areas should be designed to facilitate the establishment and maintenance of signal connectivity between the data recording unit 600 and the sensor(s) and/or first lead and second lead. The data recording unit 600 can then send the data wirelessly to a processing unit 800. Suitable data recording units are discussed hereafter.

As shown, the filled contact areas 480 are located to the periphery of the disposable absorbent article 200 and serve as connections for establishing communication between the sensor(s) and/or the first lead 470 and the second lead 475 in the area of interest 361 and the data recording unit 600. The filled contact areas 480 are in contact with the terminals 610 of the data recording unit 600. This connection is fixed so that there is a continuous flow of data from the sensor(s) and/or the first lead 470 and second lead 475 to the data recording unit.

The data recording unit 600 is an active, passive or semi passive device that may be able to interpret the signal coming from the sensor(s) and/or the first lead 470 and second lead 475 through the filled contact areas 480. Some of the exemplary functions of the data recording unit 600 are: receiving the data coming from the sensor(s) and/or the first lead 470 and second lead 475, interpret this signal, which is normally but not limited to electrical signals, into numerical values, this is normally done by an Analog to Digital Convertor (ADC), store the data and send the data to another processing unit. These functions are not necessarily always present, nor are these functions limited to the ones mentioned above. For example, the signal coming from a temperature sensor is transmitted through the first and second leads and filled contact areas of the disposable absorbent article to the data recording unit 600. The data recording unit 600 takes this signal and processes it to convert it to a digital value which then may be sent wirelessly 700 to a processing unit 800 for display. After assignation of a corresponding value to the signal, the data may be transferred from the data recording unit 600 to the processing unit 800. The transmission may occur through a wire connection using, for example, serial protocol communication or through wireless communication, for example, Bluetooth®, ZigBee®, WiFi® or NFC. However, forms of the present invention are contemplated where the data recording unit 600 is wired to the processing unit 800. Forms are also contemplated where the processing unit 800 is in direct signal communication with the filled contact areas 480.

The processing unit 800 is a device which is able to receive the data, process it and display it. This processing unit is, for example, a computer, a smartphone, a printer, a monitor or a customized active or passive device as well. Additionally, the processing unit 800 may include a desktop application which reads the data sent from the data recording unit, extract the relevant parameters and outputs a summary which contains, for example, name or number of the test or sample, date, the values relevant parameters, and a graphical representation of the obtained data. This summary is printed out though an attached printer, stored in the processing unit's memory or saved in an external device, for example, a USB stick.

The data recording unit 600 may utilize any suitable power device. For example, in some forms, the data recording unit 600 is a powered by a battery, for example, an iron-lithium battery, a lithium battery, a rechargeable nickel-metal hydride battery or a Polymer Matrix Electrolyte (PME) battery.

The disposable absorbent article is, for example, a prototype of a product, a product with enhanced properties being compared to the prior version, different types of products undergoing the same test or a group of similar products undergoing different tests. The disposable absorbent article has one or more areas of interest that may be specially examined. For example, in the course of the test method, the disposable absorbent article is laid down on a flat surface, and a liquid insult is applied to the area of interest.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for creating a disposable absorbent article for lab testing comprising the steps of:
providing a disposable absorbent article;
providing a template having a land area and an open area;
applying the template to the disposable absorbent article such that at least a portion of the disposable absorbent article is exposed through the open area, wherein the open area comprises a first open section and a second open section, the first open section and the second open section being discontinuous from one another, and wherein the open area further comprises a pair of open contact areas, one of the pair of open contact areas being associated with the first open section and another of the pair of open contact areas being associated with the second open section;
spraying a reactive composition over the template such that the reactive composition forms a first lead and a second lead, and a pair of filled contact areas on a surface of the disposable absorbent article; and
removing the template from the disposable absorbent article.

2. The method of claim 1, further comprising the step of identifying an area of interest on the disposable absorbent article.

3. The method of claim 2, wherein the open area comprises a plurality of open markers which bound the area of interest.

4. The method of claim 2, wherein the step of identifying the area of interest occurs prior to the application of the template to the disposable absorbent article.

5. The method of claim 1, wherein each of the first open section and second open section comprises at least one overlapping part, each overlapping part overlapping the area of interest.

6. The method of claim 5, wherein the first open section comprises a plurality of overlapping parts.

7. The method of claim 5, wherein each of the first open section and the second open section comprises at least two overlapping parts, each overlapping part overlapping the area of interest.

8. The method of claim 2, further comprising the step of placing a sensor in the disposable absorbent article, wherein the sensor is disposed in the area of interest.

9. The method of claim 8, wherein the sensor is selected from at least one of a relative humidity sensor, temperature sensor, blood pressure sensor, or stress sensor.

10. The method of claim 8, wherein the sensor is a temperature sensor.

11. The method of claim 1, further comprising the step of identifying a second area of interest.

12. The method of claim 11, wherein the template comprises a second open area, and wherein the second open area comprises a third open section and a fourth open section which are discontinuous with one another.

13. The method of claim 12, wherein the second open area comprises a plurality of open markers which bound the second area of interest.

14. The method of claim 11, wherein the step of identifying the second area of interest occurs prior to the application of the template to the disposable absorbent article.

15. The method of claim 12, wherein each of the third open section and fourth open section comprises at least one overlapping part, each overlapping part overlapping the second area of interest.

16. The method of claim 15, wherein the third open section comprises a plurality of overlapping parts.

17. The method of claim 15, wherein each of the third open section and fourth open section comprises at least two overlapping parts, each overlapping part overlapping the second area of interest.

18. The method of claim 11, further comprising the step of placing a sensor in the disposable absorbent article.

19. The method of any of claim 1, further comprising the step of providing a data logging device and establishing signal communication between the data logging device and the first lead and second lead.

20. The method of claim 19, wherein the method of establishing signal communication between the data recording device and the sensor comprises establishing a wireless link between the data logging device and the sensor.
